(19) United States
(12) Patent Application Publication
(10) Pub. No.: US 2008/0003191 A1
(43) Pub. Date: Jan. 3, 2008

(54) Composition combining a C-glycoside derivative and an emulsifying polymer

(75) Inventors: Jean-Thierry Simonnet, Cachan (FR); Veronique Chevalier, Villecresnes (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 11/822,107

(22) Filed: Jul. 2, 2007

Related U.S. Application Data
(60) Provisional application No. 60/836,390, filed on Aug. 9, 2006.

(30) Foreign Application Priority Data
Jul. 3, 2006 (FR) 06 06023

(51) Int. Cl. A61K 31/70; A61K 8/73

(52) U.S. Cl. 424/70.13; 514/23

(57) ABSTRACT

The present invention relates to a cosmetic and/or dermatological composition comprising, in a physiologically acceptable medium, at least one C-glycoside derivative and at least one emulsifying polymer chosen from polyolefins containing polar portion(s) and amphiphilic polymers comprising at least one acrylamido-2-methylpropanesulfonic acid (AMPS) unit.

This non provisional application claims the benefit of French Application No. 06 06023 filed on Jul. 3, 2006 and U.S. Provisional Application No. 60/836,390 filed on Aug. 9, 2006.

The present invention relates to cosmetic and/or dermatological compositions comprising at least one C-glycoside derivative and at least one emulsifying polymer. The compositions according to the invention are especially intended to improve the biological efficacy of the said derivative.

The compositions according to the invention are in particular intended for caring for and/or making up keratin materials and especially the skin.

C-glycoside derivatives are known to have interesting biological activities on the skin. C-glycoside derivatives have thus already been described in document U.S. Pat. No. 7,049,300 as having the property of stimulating the synthesis of glycosaminoglycans, thus contributing towards maintaining, inter alia, good moisturization and good suppleness of the skin.

The skin is known to constitute a natural barrier that is remarkably effective for preventing the penetration of foreign bodies and molecules into the body, but also, for example, for preventing dehydration of the body. Consequently, in order for a cosmetic product containing a C-glycoside derivative, whose target is in the live epidermis, to be effective, it is necessary for this derivative to be able to be conveyed thereto in sufficient amount while at the same time ensuring total harmlessness of the preparation on the skin, and also unquestionable cosmetic pleasantness.

Pro-penetrating agents such as Transcutol® or ethanol in high concentration are already used, especially in pharmaceuticals. However, although effective in terms of the vehicle effect, these two compounds are not entirely satisfactory as regards the other properties mentioned above.

There is thus still a need for a cosmetic composition, especially a dermatological composition, comprising a C-glycoside derivative in a sufficient amount, and which is free of the abovementioned drawbacks. Such a composition is precisely the subject of the present invention.

The inventors have in fact discovered, unexpectedly, that the combination of a C-glycoside derivative with at least one emulsifying polymer chosen from polyolefins containing polar portion(s) and amphiphilic polymers comprising at least one acrylamido-2-methylpropanesulfonic acid (AMPS) unit allows this problem to be overcome.

According to one of its first aspects, the invention thus relates to a cosmetic and/or dermatological composition comprising, in a physiologically acceptable medium, at least one C-glycoside derivative and at least one emulsifying polymer chosen from polyolefins containing polar portion(s) and amphiphilic polymers comprising at least one acrylamido-2-methylpropanesulfonic acid (AMPS) unit.

In particular, such a composition is advantageously used for caring for and/or making up keratin materials, and especially the skin.

The combination of a C-glycoside derivative with an emulsifying polymer according to the invention makes it possible to obtain cosmetic or dermatological compositions that are remarkably effective for preventing and/or combating the signs of ageing of the skin.

The term “signs of ageing of the skin” means any change in the external aspect of the skin caused by chronological or photo-induced ageing, for instance wrinkles, wizened skin, flaccid skin, thinned skin and skin lacking elasticity or tonus.

Thus, according to another of its aspects, the invention relates to method for preventing and/or combating the signs of ageing of the skin comprising at least the step of applying to the skin a combination of at least one C-glycoside derivative and of at least one emulsifying polymer chosen from polyolefins containing polar portion(s) and amphiphilic polymers comprising at least one acrylamido-2-methylpropanesulfonic acid (AMPS) unit.

The invention in particular relates to a method for making up and/or caring for keratin materials comprising at least the step of applying to the said keratin materials a combination of at least one C-glycoside derivative and of at least one emulsifying polymer chosen from polyolefins containing polar portion(s) and amphiphilic polymers comprising at least one acrylamido-2-methylpropanesulfonic acid (AMPS) unit.

Emulsifying Polymers

For the purposes of the invention, the term “emulsifying polymer” is intended to denote a polymer with amphiphilic properties, i.e. having at least one hydrophilic part and at least one hydrophobic part. Hydrophilic groups and hydrophobic groups are well known to those skilled in the art.

For the purposes of the present invention, the term “polymer” is intended to denote a compound comprising at least two repeating units and in particular at least five repeating units.

In the context of the present invention, the term “keratin materials” includes, for example, the skin and the lips.

An emulsifying polymer may especially be a polyolefin containing polar portion(s) and/or at least one amphiphilic polymer comprising at least one acrylamido-2-methyl propanesulfonic acid (AMPS) unit.

Polyolefins Containing Polar Portion(s)

The polyolefins containing polar portion(s) that may be used in the present invention are already known in other fields and are described, for example, in documents U.S. Pat. No. 5,129,972 and U.S. Pat. No. 4,919,179 as stabilizers for explosive emulsions, and in documents U.S. Pat. No. 5,518,517 and U.S. Pat. No. 5,858,055 as stabilizers for fertilizer compositions.

The polyolefins containing polar portion(s) that may be used in the compositions of the invention may comprise a polyolefinic apolar portion and at least one polar portion.

They may have a structure of block or comb type.

The polyolefinic apolar portion contains at least 40 carbon atoms and in particular from 60 to 700 carbon atoms.

This polyolefinic apolar portion may be chosen from oligomers, polymers and/or copolymers of C₂-C₂₀ monomers and especially of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins may or may not be hydrogenated.

Moreover, the polyolefins containing polar portion(s) that may be used in the compositions of the invention, for example in the form of emulsions, comprise at least one polar portion. This polar portion is advantageously present to give them amphiphilic properties.

Thus, these polyolefins containing polar portion(s) can lower the interface tension (water/oil) by at least 10 mN/m when they are present at a concentration of 0.01% by weight relative to the total weight of the oily phase. For example, the polyolefin with succinic end groups sold under the name Lubrizol 2724® by the company Lubrizol, at a concentration of 0.01% by weight relative to the total weight of the oily phase, lowers the interface tension by 15 mN/m at the interface of an aqueous phase consisting of an aqueous 1% MgSO₄ solution and of an oily phase comprising a mixture of oils (isohexadecane/hydrogenated polyisobutene/volatile silicone in an 8/6/4 ratio), the ratio of the aqueous phase and of the oily phase being equal to 1.

The polar portion of the polyolefins containing polar portion(s) of the invention may be anionic, cationic, nonionic, zwitterionic or amphoteric. It may consist, for example, of polyalkylene glycols, polyalkyleneimines, carboxylic or dicarboxylic acids, anhydrides thereof or derivatives thereof such as the esters, amides and salts thereof.

The polyolefins containing carboxylic acid polar portion(s) may be derived, for example, from the reaction between a polyolefin and at least one carboxylic acid or anhydride, which is optionally totally or partially salified, chosen from the group comprising succinic acid or anhydride, maleic acid, maleic anhydride, fumaric acid, itaconic acid, citraconic acid (or methylmaleic acid), mesaconic acid (or methylfumaric acid) and aconitic acid, ester or amide derivatives thereof, and mixtures thereof.

According to one embodiment, the polar portion of the polyolefin may be chosen from the group comprising polyoxyethylene, succinic acid or anhydride, esters or amides of succinic acid or anhydride, alkali metal or alkaline-earth metal salts or organic salts of succinic acid or anhydride, or partial salts of succinic acid or anhydride monoesters or monoamides.

The polyolefins containing polyoxyethylene polar portion(s) may be chosen, for example, from polyisoprene-polyoxyethylene diblock polymers and poly(ethylene-co-propylene)-polyoxyethylene polymers, and mixtures thereof. These polymers are especially described in the publication by Allgaier et al., Macromolecules, 1997, vol. 30, pp. 1582-1586.

The polyolefins containing succinic acid or anhydride polar portion(s) may be chosen especially from the polyolefin derivatives of succinic acid or anhydride described in U.S. Pat. No. 4,234,435, U.S. Pat. No. 4,708,753, U.S. Pat. No. 5,129,972, U.S. Pat. No. 4,931,110, GB-A-2 156 799, U.S. Pat. No. 4,877,756 and U.S. Pat. No. 4,919,179.

The polyolefinic portion may consist, for example, of hydrogenated or non-hydrogenated polyisobutylene, with a molecular weight ranging from 400 to 5000 g/mol. In the polyisobutylene containing succinic end groups thus obtained, the succinic part may be optionally modified, i.e. esterified, amidated or in salt form. It may be modified with alcohols, amines, alkanolamines or polyols, or may be in the form of salts of alkali metal or of alkaline-earth metal, of ammonium or of an organic base, for instance the diethanolamine, triethanolamine or diethylethanolamine salts.

The polyolefins containing esterified or amidated succinic end groups are reaction products of (a) a polyolefin containing succinic end groups and of (b) an amine or an alcohol, to form an amide or an ester.

The term “amine” used herein comprises all types of amine, including alkanolamines. They may be, for example, primary, secondary or tertiary monoamines, these amines possibly being aliphatic, cycloaliphatic, aromatic or heterocyclic, and saturated or unsaturated.

Examples of alkanolamines that may especially be mentioned include diethylethanolamine and triethanolamine.

Moreover, the alcohols may be monoalcohols or polyalcohols.

The monoalcohols comprise primary, secondary or tertiary aliphatic alcohols, and phenols.

The polyalcohols may be chosen, for example, from aliphatic, cycloaliphatic, aromatic and heterocyclic polyalcohols.

An example of a polyalcohol that may especially be mentioned is glycerol.

The polyolefins containing modified (esterified or amidated) succinic end groups and the process for preparing them are described in particular in document U.S. Pat. No. 4,708,753. Polyolefins containing esterified succinic end groups are preferably used.

Polyolefins containing succinic end groups that may especially be mentioned include polyisobutylenes containing succinic end groups that are modified, especially esterified, for example with diethanolamine, and salts thereof, especially the diethanolamine salts, such as the products sold under the names Lubrizol® 2724, Lubrizol® 2722 and Lubrizol® 5603 by the company Lubrizol.

Another example of a polyolefin containing polar portions that may be used in the invention is the product of reaction of maleic anhydride with polyisobutylene, such as the products sold under the name Glissopal® (Glissopal® 2300, 1300 and 1000) (INCI name: polyisobutene) by the company BASF.

The polyolefin containing polar portion(s) that is particularly preferred is a product of reaction of polyisobutylenylsuccinic anhydride with diethylethanolamine, thus forming a diethylethanolamine salt of polybutene 2-(N,N-diethyl)aminoethyl succinate.

This product is sold, for example, under the name Lubrizol® 5603 by the company Lubrizol, and may be represented by the following formula:

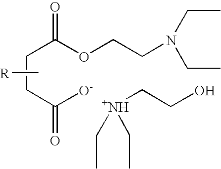

in which R represents a polyisobutenyl group, especially with a weight-average molecular mass of 1000 g/mol. This product has the INCI name: hydroxyethyldiethonium polyisobutenyl triethylaminosuccinate (and) diethylethanolamine.

Another polyolefin containing polar portion(s) that is particularly envisaged is a polyisobutenyl succinate of diethanolaminoethyl and of triethanolamine. This product is sold, for example, under the name Chemcinnate® 2000 by the company Chemron.

A polyolefin containing polar portion(s) that may also be used is a polyisobutenyl glyceryl succinate ester, especially the product sold under the name Chemcinnate® 1000 AF by the company Chemron.

The compositions according to the invention may comprise from 0.01% to 10% by weight, in particular from 0.1% to 7% by weight and better still from 0.2% to 5% by weight of polyolefin(s) containing polar portion(s) relative to the total weight of the composition.

The polyolefins containing polar portion(s) according to the invention may be used, for example, as additive in an emulsion, and in this case may be dissolved in the oily phase thereof.

They may also be used as emulsifier and allow the formation of water-in-oil (W/O) emulsions as described in patent application FR 2 811 565.

Amphiphilic Polymers Comprising at Least One acrylamido-2-methyl-propanesulfonic Acid (AMPS) Unit

The amphiphilic polymers comprising at least one acrylamido-2-methyl-propanesulfonic acid (AMPS) unit that may be used in the present invention, which are also known more simply as “amphiphilic AMPS polymers” hereinbelow, comprise both a hydrophilic part and a hydrophobic part comprising at least one fatty chain.

The fatty chain present in the said amphiphilic AMPS polymers according to the invention may preferably contain from 7 to 30 carbon atoms and more preferentially from 7 to 22 carbon atoms.

The amphiphilic AMPS polymers according to the invention are especially chosen from amphiphilic polymers of at least one acrylamidomethylpropanesulfonic acid (AMPS) monomer and of at least one ethylenically unsaturated comonomer comprising at least one hydrophobic part containing from 7 to 30 carbon atoms and in particular from 7 to 22 carbon atoms or even from 12 to 22 carbon atoms.

The amphiphilic AMPS polymers according to the invention generally have a weight-average molecular weight ranging from 50 000 to 10 000 000 g/mol, in particular from 100 000 to 8 000 000 g/mol and even more particularly from 100 000 to 7 000 000 g/mol.

They may be crosslinked or non-crosslinked.

When the amphiphilic AMPS polymers according to the invention are crosslinked, the crosslinking agents may be chosen from the polyolefinically unsaturated compounds commonly used for the crosslinking of polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebis(acrylamide), methylenebis(methacrylamide), triallylamine, triallyl cyanurate, diallyl maleate, tetraallylethylenediamine, tetraallyloxyethane, trimethylolpropane diallyl ether, allyl(meth)acrylate, allylic ethers of alcohols of the sugar series, or other allylic or vinyl ethers of polyfunctional alcohols, and also allylic esters of phosphoric and/or vinylphosphonic acid, or mixtures of these compounds.

The crosslinking agents may be chosen especially from methylenebis(acrylamide), allyl methacrylate and trimethylolpropane triacrylate (TMPTA).

The degree of crosslinking may range, for example, from 0.01 mol % to 10 mol % and preferably from 0.2 mol % to 2 mol % relative to the polymer.

The amphiphilic AMPS polymers according to the invention may be chosen especially from statistical amphiphilic AMPS polymers modified by reaction with a C₆-C₂₂ n-monoalkylamine or di-n-alkylamine such as those described in patent application WO 00/31154.

An amphiphilic polymer that is suitable for use in the invention may comprise at least one ethylenically unsaturated hydrophilic monomer chosen, for example, from acrylic acid, methacrylic acid or substituted alkyl derivatives thereof or esters thereof obtained with monoalkylene or polyalkylene glycols, acrylamide, methacrylamide, vinylpyrrolidone, vinylformamide, maleic anhydride, itaconic acid or maleic acid, or mixtures thereof.

An amphiphilic polymer according to the invention may comprise at least one ethylenically unsaturated hydrophobic comonomer.

An amphiphilic polymer that is suitable for use in the invention may comprise at least one hydrophobic part chosen from saturated or unsaturated, linear alkyl radicals, for instance n-octyl, n-decyl, n-hexadecyl, n-dodecyl and oleyl, branched alkyl radicals, for instance isostearyl, or cyclic alkyl radicals, for instance cyclododecane or adamantane.

An amphiphilic AMPS polymer may also contain at least one ethylenically unsaturated hydrophobic comonomer comprising, for example:
a fluoro or C₇-C₁₈ fluoroalkyl radical (for example the group of formula —(CH₂)₂—(CF₂)₉—CF₃),
a cholesteryl radical or a cholesterol-based radical (for example cholesteryl hexanoate),
a polycyclic aromatic group, for instance naphthalene or pyrene,
a silicone, alkylsilicone or alkylfluorosilicone radical.

These copolymers are especially described in document EP-A-750 899, patent U.S. Pat. No. 5,089,578 and in the following publications by Yotaro Morishima:
“Self-assembling amphiphilic polyelectrolytes and their nanostructures—Chinese Journal of Polymer Science Vol. 18, No. 40, (2000), 323-336”;
“Micelle formation of random copolymers of sodium 2-(acrylamido)-2-methylpropanesulfonate and a nonionic surfactant macromonomer in water as studied by fluorescence and dynamic light scattering—Macromolecules 2000, Vol. 33, No. 10-3694-3704”;
“Solution properties of micelle networks formed by nonionic moieties covalently bound to a polyelectrolyte: salt effects on rheological behavior—Langmuir, 2000, Vol. 16, No. 12, 5324-5332”;
“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

They are also described in documents EP 1 069 142, WO 02/44224, WO 02/44225, WO 02/44227, WO 02/44229, WO 02/44230, WO 02/44231, WO 02/44267, WO 02/44268, WO 02/44269, WO 02/44270, WO 02/44271, WO 02/43677, WO 02/43686, WO 02/43687, WO 02/43688 and WO 02/43689, in the name of Clariant.

An ethylenically unsaturated hydrophobic comonomer of the invention may preferably be chosen from the acrylates or acrylamides of formula (1) below: 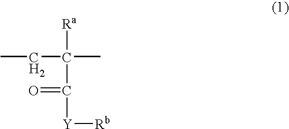
in which:
R^{a} denotes a hydrogen atom or a linear or branched C₁-C₆ alkyl radical, preferably methyl;
Y denotes O or NH;
R^{b} denotes a hydrophobic radical comprising a fatty chain containing from 7 to 30 carbon atoms, preferably from 7 to 22 and more particularly from 12 to 22 carbon atoms.

The hydrophobic radical R^{b} is chosen from saturated or unsaturated linear C₇-C₂₂ alkyl radicals (for example n-octyl, n-decyl, n-hexadecyl, n-dodecyl or oleyl), branched alkyl radicals (for example isostearic) or cyclic alkyl radicals (for example cyclo-dodecane or adamantane); C₇-C₁₈ alkylperfluoro radicals (for example the group of formula —(CH₂)₂—(CF₂)₉—CF₃); the cholesteryl radical or a cholesterol ester, for instance cholesteryl hexanoate; aromatic polycyclic groups, for instance naphthalene or pyrene.

Among these radicals, linear and branched alkyl radicals are more particularly preferred.

According to one preferred form of the invention, the hydrophobic radical R^{b} may also comprise at least one alkylene oxide unit and preferably a polyoxyalkylene chain.

The polyoxyalkylene chain may preferentially consist of ethylene oxide units and/or propylene oxide units and even more particularly consists solely of ethylene oxide units.

The number of moles of oxyalkylene units may generally range from 1 to 30 mol, more preferably from 1 to 25 mol and even more preferably from 3 to 20 mol.

Among these polymers that may be mentioned are:
crosslinked or non-crosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl(meth)acrylate units relative to the polymer, such as those described in patent application EP-A-750 899;
terpolymers comprising from 10 mol % to 90% of acrylamide units, from 0.1 mol % to 10 mol % of AMPS units and from 5 mol % to 80 mol % of n-(C₆-C₁₈)alkylacrylamide units relative to the polymer, such as those described in U.S. Pat. No. 5,089,578;
partially or totally neutralized non-crosslinked copolymers of AMPS and of n-dodecyl, n-hexadecyl or n-octadecyl methacrylate, such as those described in the Morishima articles mentioned above;
non-crosslinked and crosslinked copolymers of partially or totally neutralized AMPS and of n-dodecylmethacrylamide, such as those described in the Morishima articles mentioned above.

Amphiphilic AMPS polymers that may also be mentioned include copolymers of totally neutralized AMPS and of n-dodecyl, n-hexadecyl and/or n-octadecyl methacrylate, and also non-crosslinked and crosslinked copolymers of AMPS and of n-dodecylmethacrylamide.

Mention will be made more particularly of crosslinked or non-crosslinked amphiphilic AMPS copolymers consisting of:

(a) 2-acrylamido-2-methylpropanesulfonic acid (AMPS) units of formula (2) below: 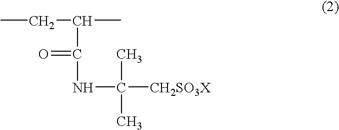
in which X is a proton, an alkali metal cation, an alkaline-earth metal cation or an ammonium ion;

(b) and units of formula (3) below: 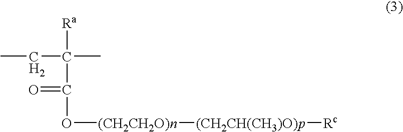
in which n and p, independently of one another, denote a number of moles and range from 0 to 30, preferably from 1 to 25 and more preferably from 3 to 20, with the proviso that n+p is less than or equal to 30, preferably less than 25 and better still less than 20; R^{a} denotes a hydrogen atom or a linear or branched C₁-C₆ alkyl radical, preferably methyl, and R^{c} denotes a linear or branched alkyl containing from 7 to 22 and preferably from 12 to 22 carbon atoms.

In formula (2), the cation X more particularly denotes sodium or ammonium.

Among the monomers of formula (3) that may be mentioned are:
esters of (meth)acrylic acid and of a C₁₀-C₁₈ fatty alcohol polyoxyethylenated with 8 EO, for instance the product Genapol C-080® sold by the company Clariant,
esters of (meth)acrylic acid and of a C₁₁ fatty oxo alcohol polyoxyethylenated with 8 EO, for instance the product Genapol UD-080® sold by the company Clariant,
esters of (meth)acrylic acid and of a C₁₂-C₁₄ polyoxyethylenated fatty alcohol with 7 EO, for instance the product Genapol LA-070® sold by the company Clariant,
esters of (meth)acrylic acid and of a C₁₂-C₁₄ polyoxyethylenated fatty alcohol with 11 EO, for instance the product Genapol LA-110® sold by the company Clariant,
esters of (meth)acrylic acid and of a C₁₆-C₁₈ polyoxyethylenated fatty alcohol with 8 EO, for instance the product Genapol T-080® sold by the company Clariant,
esters of (meth)acrylic acid and of a C₁₆-C₁₈ polyoxyethylenated fatty alcohol with 15 EO, for instance the product Genapol T-150® sold by the company Clariant,
esters of (meth)acrylic acid and of a C₁₆-C₁₈ polyoxyethylenated fatty alcohol with 11 EO, for instance the product Genapol T-110® sold by the company Clariant,
esters of (meth)acrylic acid and of a C₁₆-C₁₈ polyoxyethylenated fatty alcohol with 20 EO, for instance the product Genapol T-200® sold by the company Clariant,
esters of (meth)acrylic acid and of a C₁₆-C₁₈ polyoxyethylenated fatty alcohol with 25 EO, for instance the product Genapol T-250® sold by the company Clariant,
esters of (meth)acrylic acid and of a C₁₈-C₂₂ polyoxyethylenated fatty alcohol with 25 EO and/or of a C₁₆-C₁₈ polyoxyethylenated fatty isoalcohol with 25 EO.

The products that will be chosen more particularly are:

(i) non-crosslinked products for which p=0, n=7 or 25, R^{a} denotes methyl and R^{c} represents a C₁₂-C₁₄ or C₁₆-C₁₈ alkyl mixture,

(ii) crosslinked products for which p=0, n=8 or 25, R^{a} denotes methyl and R^{c} represents a C₁₆-C₁₈ alkyl mixture.

These polymers are described and synthesized in patent application EP 1 069 142.

These particular amphiphilic AMPS polymers may be obtained according to the standard processes of free-radical polymerization in the presence of one or more initiators, for instance azobisisobutyronitrile (AIBN), azobisdimethylvaleronitrile, 2,2-azobis(2-amidinopropane) hydrochloride (ABAH), organic peroxides such as dilauryl peroxide, benzoyl peroxide, tert-butyl hydroperoxide, etc., mineral peroxide compounds such as potassium or ammonium persulfate, or H₂O₂ optionally in the presence of reducing agents.

These amphiphilic AMPS polymers may be obtained especially by free-radical polymerization in tert-butanol medium, in which they precipitate. By using precipitation polymerization in tert-butanol, it is possible to obtain a size distribution of the polymer particles that is particularly favourable for its uses.

The reaction may be performed at a temperature of between 0 and 150° C. and preferably between 10 and 100° C., either at atmospheric pressure or under reduced pressure.

It may also be performed under inert atmosphere and preferably under nitrogen.

The amphiphilic AMPS polymers according to the invention may preferably be partially or totally neutralized with a mineral base such as sodium hydroxide, potassium hydroxide, aqueous ammonia or an organic base such as monoethanolamine, diethanolamine, triethanolamine, an aminomethylpropanediol, N-methylglucamine, basic amino acids, for instance arginine and lysine, and mixtures of these compounds. They may especially be totally or almost totally neutralized, i.e. at least 80% neutralized.

The molar percentage concentration of the units of formula (2) and of the units of formula (3) in the amphiphilic AMPS polymers according to the invention may vary as a function of the desired cosmetic application, the nature of the emulsion (oil-in-water or water-in-oil emulsion) and the rheological properties of the desired formulation. It can for example range between 0.1 and 99.9 mol %.

The amphiphilic AMPS polymers according to the invention that are sparingly hydrophobic will be more suitable for thickening and/or stabilizing oil-in-water emulsions.

The molar proportion of units of formula (3) may preferably range from 0.1% to 50%, more particularly from 1% to 25% and even more particularly from 3% to 10%.

The amphiphilic AMPS polymers according to the invention that are more hydrophobic will be more suitable for thickening and/or stabilizing water-in-oil emulsions.

The molar proportion of units of formula (3) will preferably range from 50.1% to 99.9%, more particularly from 60% to 95% and even more particularly from 65% to 90%.

The distribution of the monomers in the amphiphilic AMPS polymers of the invention may be, for example, alternate, block (including multiblock) or random.

As a guide, and without this being limiting, mention may be made especially of the copolymer of AMPS and of ethoxylated C₁₂-C₁₄ alcohol methacrylate (non-crosslinked copolymer obtained from Genapol LA-070 and from AMPS) (CTFA name: Ammonium Acryloyldimethyltaurate/Laureth-7 methacrylate copolymer) sold under the name Aristoflex LNC by the company Clariant, the copolymer of AMPS and of ethoxylated (25 EO) stearyl methacrylate (copolymer crosslinked with trimethylolpropane triacrylate, obtained from Genapol T-250 and from AMPS) (CTFA name: Ammonium Acryloyldimethyltaurate/Steareth-25 Methacrylate Crosspolymer) sold under the name Aristoflex HMS by the company Clariant, Aristoflex SNC (80/20 copolymer of AMPS/ethoxylated (8 mol EO) C₁₆-C₁₈ alcohol methacrylate; CTFA name: Ammonium Acryloyldimethyltaurate/Steareth-8 methacrylate copolymer) and Aristoflex HMB (copolymer of AMPS/ethoxylated (25 EO) behenyl methacrylate, crosslinked with trimethylolpropane triacrylate (TMPTA)).

The amphiphilic AMPS polymers according to the invention may be present in active material amounts ranging from 0.01% to 20% by weight, more preferentially from 0.1% to 10% by weight, even more preferentially from 0.1% to 5% by weight and even more particularly from 0.3% to 2% by weight relative to the total weight of the composition.

The amphiphilic AMPS polymers according to the invention may be used as additive in an emulsion, and in this case may preferentially be dissolved in the aqueous phase thereof.

They may also be used as emulsifier, and may allow the formation of an oil-in-water (O/W) emulsion.

C-Glycoside Derivatives

A C-glycoside derivative that is suitable for use in the invention may be a compound of general formula (I) below: 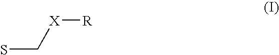
in which:
R represents:
   a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
   a saturated C₁-C₂₀ and in particular C₁-C₁₀ or unsaturated C₂-C₂₀ and in particular C₂-C₁₀, or saturated or unsaturated, branched or cyclic C₃-C₂₀ and in particular C₃-C₁₀ linear hydrofluoroalkyl or perfluoroalkyl radical;
      the hydrocarbon-based chain constituting the said radicals possibly being, where appropriate, interrupted with 1, 2, 3 or more heteroatoms chosen from:
an oxygen,
a sulfur,
a nitrogen, and
a silicon,
   and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
   with R₄ and R₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ linear alkyl, perfluoroalkyl or hydrofluoroalkyl radical; or a C₆-C₁₀ aryl radical,
X represents a radical chosen from the groups: 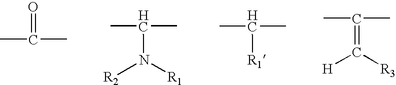
   with R₁, R₂ and R₃ representing, independently of each other, a hydrogen atom or a radical R, with R as defined above, and R′₁ represents a hydrogen atom, an —OH group or a radical R as defined above, R₁ possibly also denoting a C₆-C₁₀ aryl radical;
S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be α or β, and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, the said monosaccharide or polysaccharide containing at least one hydroxyl function that is mandatorily free and/or optionally one or more amine functions that are mandatorily protected, X and R otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)— and —CH(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (I) for which R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X otherwise conserving all the definitions given above. Preferably, R denotes a linear C₁-C₄ and especially C₁-C₃ radical optionally substituted with —OH, —COOH or —COOR″₂, R″₂ being a saturated C₁-C₄ alkyl radical, especially ethyl.

Preferentially, R denotes an unsubstituted linear C₁-C₄ and especially C₁-C₂ alkyl radical, in particular ethyl.

Among the C-glycoside derivatives of formula (I) that are preferably used are those for which:
R represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X represents —CO—, —CH(OH)—, —CH(NR₁R₂)— or —CH(R)—, as defined above.

Preferably, a C-glycoside derivative of formula (I) is used, for which:
R denotes a linear C₁-C₄ and especially C₁-C₃ radical, optionally substituted with —OH, —COOH or —COOR″₂, R″₂ being a saturated C₁-C₄ alkyl radical, especially ethyl;
S represents a monosaccharide as described above;
X represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group.

Preferentially, a C-glycoside derivative of formula (I) is used, for which:
R denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X represents a group chosen from —CO—, —CH(OH)— and —CH(NH₂)— and preferentially a —CH(OH)— group.

The salts that are acceptable for the non-therapeutic use of the compounds described in the present invention comprise conventional non-toxic salts of the said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (I) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates such as those formed during the final step of preparation of the said compounds due to the presence of solvents. Examples that may be mentioned include the solvates due to the presence of water or of linear or branched alcohols, for instance ethanol or isopropanol.

Among the C-glycoside derivatives of formula (1) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-α-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C—O-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl 3-methyl-4-(acetamido-C-β-D-glucopyranosyl)butyrate;
152. ethyl 3-methyl-4-(acetamido-C-α-D-glucopyranosyl)butyrate;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As non-limiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may advantageously be used for the preparation of a composition according to the invention.

According to one particular embodiment, the C-glycoside derivative is C-β-D-xylopyranoside-2-hydroxypropane in the form of a solution containing 30% active material in a water/propylene glycol mixture (60%/40% by weight) such as the product manufactured by Chimex under the trade name Mexoryl SBB®.

Needless to say, according to the invention, a C-glycoside derivative corresponding to formula (I) may be used alone or as a mixture with other C-glycoside derivatives and in all proportions.

A C-glycoside derivative that is suitable for use in the invention may especially be obtained via the synthetic method described in document WO 02/051 828.

The amount of C-glycoside derivative to be used in a composition according to the invention depends on the desired cosmetic or therapeutic effect, and may thus vary within a wide range.

A person skilled in the art can readily determine the appropriate amounts, on the basis of his general knowledge.

A composition according to the invention may comprise from about 0.0001% to about 25% by weight of active material relative to the total weight of the composition, in particular from about 0.001% to about 10% by weight of active material and more particularly from about 0.05% to about 5% by weight of C-glycoside derivative relative to the total weight of the composition.

A composition in accordance with the invention comprises a physiologically acceptable medium.

This physiologically acceptable medium may at least comprise an aqueous phase optionally as a mixture with one or more organic solvents such as a C₁-C₈ alcohol, especially ethanol, isopropanol, tert-butanol, n-butanol, polyols, for instance glycerol, propylene glycol or butylene glycol, and polyol ethers.

A composition according to the invention may be anhydrous.

The term “anhydrous composition” refers to any composition comprising less than 5% water and more preferentially less than 1% water relative to the total weight of the composition.

A composition according to the invention may also comprise a fatty phase, which may comprise oils, gums or waxes usually used in the field of application under consideration.

Thus, according to one embodiment, a composition according to the invention may also comprise at least one fatty phase chosen from a fatty phase that is solid at room temperature (20-25° C.) and atmospheric pressure, a liquid fatty phase, and a mixture thereof.

A liquid fatty phase that is suitable for use in the invention may comprise a volatile oil, a non-volatile oil, and a mixture thereof. A volatile or non-volatile oil may be a hydrocarbon-based oil, especially of animal or plant origin, a synthetic oil, a silicone oil, a fluoro oil, or a mixture thereof.

A solid fatty phase that is suitable for use in the invention may be chosen, for example, from pasty fatty substances and gums, and mixtures thereof.

As oils or waxes that may be used in the invention, mention may be made of mineral oils (liquid petroleum jelly), plant oils (liquid fraction of shea butter, sunflower oil), animal oils (perhydrosqualene), synthetic oils (purcellin oil), silicone oils or waxes (cyclomethicone) and fluoro oils (perfluoropolyethers), and beeswax, carnauba wax or paraffin wax. Fatty alcohols and fatty acids (stearic acid) may be added to these oils.

When a composition is an emulsion, the proportion of the fatty phase may range from 5% to 80% by weight and preferably from 5% to 50% by weight relative to the total weight of the composition. The oils, waxes, emulsifiers and coemulsifiers, other than the emulsifying polymers used in the present patent application, which may be used in the composition in emulsion form are chosen from those conventionally used in cosmetics.

Besides the emulsifying polymers according to the invention, the compositions according to the invention may comprise from 0.3% to 30% by weight and in particular from 0.5% to 20% by weight of emulsifier(s) and/or coemulsifier(s) relative to the total weight of the composition.

A composition according to the invention may also contain lipid vesicles.

When a composition according to the invention is an oily solution or gel, the fatty phase may represent more than 90% of the total weight of the composition.

A composition according to the invention may also contain adjuvants that are common in the field under consideration, such as other surfactants or emulsifiers, hydrophilic or lipophilic gelling agents, hydrophilic or lipophilic additives, preserving agents, antioxidants, solvents, fragrances, fillers, UVA and/or UVB screening agents (organic or mineral), pigments, fibres, chelating agents, odour absorbers, dyestuffs, and other cosmetic or pharmaceutical active agents.

The amounts of these various adjuvants are those conventionally used in cosmetics, and may be, for example, from 0.01% to 10% of the total weight of the composition. Depending on their nature, these adjuvants may be introduced into the fatty phase, into the aqueous phase and/or into lipid spherules.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and as lipophilic gelling agents that may be used, mention may be made of modified clays, for instance bentones, metal salts of fatty acids, for instance aluminium stearates, hydrophobic silica and polyethylene.

A composition of the invention may be in any conceivable galenical form.

In particular, a composition according to the invention may have the form of an aqueous, alcoholic or aqueous-alcoholic solution; a dispersion of the lotion or serum type; a water-in-oil, oil-in-water or multiple emulsion; a suspension; microcapsules or microparticles; vesicular dispersions of ionic and/or nonionic type; an aqueous or oily lotion or a lotion in serum form; capsules, granules, syrups or tablets; a mousse or a solid preparation; an aerosol composition also comprising a pressurized propellant.

A composition according to the invention may be in the form of a haircare composition, especially a shampoo, a hairsetting lotion, a medicated lotion, a styling cream or gel, a dye composition, especially for oxidation dyeing, restructuring lotions for the hair, a permanent-waving composition (especially a composition for the first stage of a permanent-waving operation), a lotion or gel for preventing hair loss, or an antiparasitic shampoo.

It may also be in the form of a cleansing, protective, treating or care composition for the face, the hands, the feet, the major anatomical folds or the body (for example a day cream, a night cream, a makeup-removing cream, an antisun composition, a protective or care body milk, an after-sun milk, a skincare lotion, gel or mousse, for example a cleansing lotion, or an artificial tanning composition); a facial or body makeup composition such as a foundation; a bath composition; a deodorizing composition comprising, for example, a bactericidal agent; an aftershave composition, a hair-removing composition; an insect-repelling composition; a pain-relief composition; a composition for treating certain skin diseases, for instance eczema, rosacea, psoriasis, lichens and severe pruritus.

When a composition according to the invention is intended for a use of scrubbing type, it may also be in any galenical form mentioned above, provided that it can be removed easily by rinsing, especially in the form of an aqueous gel or an aqueous or aqueous-alcoholic solution.

A composition according to the invention may be applied by any means that allows uniform distribution, and especially using cotton wool, a cotton tip, a brush, a gauze, a spatula or a pad, or alternatively by spraying, and may be removed by rinsing with water or using a mild detergent.

A composition according to the invention may also comprise one or more additional cosmetic or therapeutic active agent(s), for instance anti-ageing/antiwrinkle agents (such as anti-glycation agents for stimulating the synthesis of dermal or epidermal macromolecules and/or for preventing their degradation, for stimulating fibroblast and/or keratinocyte proliferation or for stimulating keratinocyte differentiation, and muscle relaxants), moisturizers, desquamating agents, antipollution agents and free-radical scavengers, slimming agents, agents acting on the capillary circulation, agents acting on the energy metabolism of cells, tensioning agents, depigmenting or propigmenting agents, desquamating agents, antiacne agents or anti-inflammatory/anti-irritant agents.

Mention may also be made of any active agent known for its activity on ageing of the skin, for instance keratolytic or pro-desquamating agents, for example α-hydroxy acids, β-hydroxy acids, α-keto acids, retinoids and esters thereof, retinol, and retinoic acid and derivatives thereof, vitamins C, B3 or PP, B5 and E and derivatives of these vitamins and especially esters thereof, vitamin K and derivatives thereof (K1, K2, etc.); free-radical scavengers; DHEA and derivatives thereof; coenzyme Q10; bleaching and depigmenting agents, for instance kojic acid, para-aminophenol derivatives, arbutin and derivatives thereof, and mixtures thereof.

A composition according to the invention may be intended for cosmetic care and/or makeup and/or dermatological use.

The examples below are given as illustrations of the invention and should not be interpreted as limiting its scope.

### EXAMPLES

The C-glycoside derivative used is C-β-D-xylopyranoside-2-hydroxypropane, sold under the name Mexoryl SBB® by Chimex. It is in the form of a solution containing 30% by weight of active material (AM) in a 60/40 water/1,2-propanediol mixture.

### Example 1

Aqueous solution according to the invention comprising a C-glycoside derivative and, as emulsifying polymer, an amphiphilic AMPS polymer according to the invention.

| | |
|---|---|
| 80/20 AMPS/ethoxylated (8 mol EO) C₁₂-C₂₄ alcohol | 1% |
| methacrylate copolymer (Aristoflex LNC ® from Clariant) | |
| Distilled water | qs 100% |
| C-β-D-xylopyranoside-2-hydroxypropane | 2% (AM) |

Comparative example:

| | |
|---|---|
| Distilled water | qs 100% |
| C-β-D-xylopyranoside-2-hydroxypropane | 2% (AM) |

The analysis method used is that described in Example 10 of patent application WO 02/051 828 (three-dimensional model of artificial skin).

The neosynthesis of glycosaminoglycans is significantly greater using the composition of Example 1 than with that described in the comparative example.

### Example 2

Oil-in-water emulsion comprising a C-glycoside derivative and, as emulsifying polymer, an amphiphilic AMPS polymer according to the invention.

Phase 1

| | |
|---|---|
| Apricot kernel oil | 7% |
| Isononyl isononanoate | 7% |
| Cyclomethicone | 7% |
| Fragrance | 0.5% |

Phase 2

| | |
|---|---|
| Distilled water | qs 100% |
| C-β-D-xylopyranoside-2-hydroxypropane | 5% (AM) |
| 80/20 AMPS/ethoxylated (8 mol EO) C₁₂-C₂₄ alcohol | 1% |
| methacrylate copolymer (Aristoflex LNC ® from Clariant) | |
| Glyceryl isostearate | 1% |
| Preserving agents | 0.5% |

AM: active material
Phase 1 is added to phase 2 in which the product Aristoflex LNC has previously been hydrated, at room temperature, and with vigorous stirring using a rotor-stator, for 30 minutes.
The composition thus obtained is in the form of a very fine, stable water-in-oil emulsion.

### Example 3

Water-in-oil emulsion comprising a C-glycoside derivative and, as emulsifying polymer, a polyolefin containing polar portion(s).

Phase 1

| | |
|---|---|
| Isohexadecane | 7% |
| Cyclopentasiloxane | 5% |
| Isododecane | 2% |
| Mixture of polyisobutenyl triethanolamine-diethanolamine | 3.5% |
| succinate and of 2-ethylhexyl palmitate (sold under the | |
| reference Chemcinnate 2000 ® by the company Chemron) | |
| Preserving agent | 0.3% |

Phase 2

| | |
|---|---|
| C-β-D-xylopyranoside-2-hydroxypropane | 5% AM |
| Magnesium sulfate | 0.8% |
| Preserving agents | 0.5% |
| Water | qs 100% |

AM: active material
Phase 2 is introduced into phase 1 with vigorous stirring.
The composition thus obtained is in the form of a fluid water-in-oil emulsion.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic and/or dermatological composition comprising, in a physiologically acceptable medium, at least one C-glycoside derivative and at least one emulsifying polymer chosen from polyolefins containing polar portion(s) and amphiphilic polymers comprising at least one acrylamido-2-methylpropanesulfonic acid (AMPS) unit.

2. Composition according to claim 1, in which the C-glycoside derivative corresponds to the general formula (I) below: 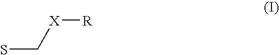
in which:
R represents:
a saturated C₁-C₂₀ or unsaturated C₂-C₂₀linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR₄,
—SR₄,
—NR₄R₅,
—COOR₄,
—CONHR₄,
—CN,
a halogen atom,
a C₁-C₆ hydrofluoroalkyl or perfluoroalkyl radical, and/or
a C₃-C₈ cycloalkyl radical,
with R₄ and R₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X represents a radical chosen from the groups: 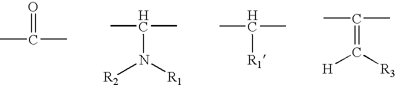
with R₁, R₂ and R₃ representing, independently of each other, a hydrogen atom or a radical R, with R as defined above, and R′₁ representing a hydrogen atom, an —OH group or a radical R as defined above, R₁ possibly also denoting a C₆-C₁₀ aryl radical;
S represents a monosaccharide or a polysaccharide comprising up to 20 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X represents a bond of C-anomeric nature, which may be α or β, and also the cosmetically acceptable salts thereof, the solvates thereof such as hydrates, and the isomers thereof.

3. Composition according to claim 2, in which S represents a monosaccharide chosen from D-glucose, D-xylose, L-fucose, D-galactose and D-maltose.

4. Composition according to claim 2, in which X represents a group chosen from —CO—, —CH(OH)— and —CH(NH₂)— group.

5. Composition according to claim 2, in which R denotes a linear C₁-C₄ C₁-C₃ radical, optionally substituted with —OH, —COOH or —COOR″₂, R″₂ being a saturated C₁-C₄ alkyl radical.

6. Composition according to claim 1, in which the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

7. Composition according to claim 1, in which the C-glycoside derivative is chosen from C-β-D-xylopyranoside-2-hydroxypropane and C-α-D-xylopyranoside-2-hydroxypropane.

8. Composition according to claim 1, comprising the C-glycoside derivative in a proportion of from about 0.00001% to about 25% by weight of active material relative to the total weight of the composition.

9. Composition according to claim 1, in which the emulsifying polymer is a polyolefin containing polar portion(s).

10. Composition according to claim 9, in which the polyolefin containing polar portion(s) comprises a polyolefinic apolar portion containing at least 40 carbon atoms and in particular from 60 to 700 carbon atoms.

11. Composition according to claim 10, in which the polyolefinic apolar portion is chosen from oligomers, polymers and/or copolymers of C₂-C₂₀ monomers.

12. Composition according to claim 11, in which the polyolefinic apolar portion is chosen among ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene.

13. Composition according to claim 9, in which the polar portion of the polyolefin is anionic, cationic, nonionic, zwitterionic or amphoteric.

14. Composition according to claim 9, in which the polar portion of the polyolefin consists of polyalkylene glycols, polyalkyleneimines, carboxylic or dicarboxylic acids, anhydrides thereof or derivatives thereof and salts thereof.

15. Composition according to claim 9, in which the polar portion of the polyolefin is chosen from the group comprising polyoxyethylene, succinic acid or anhydride, esters or amides of succinic acid or anhydride, alkali metal or alkaline-earth metal salts or organic salts of succinic acid or anhydride, or the partial salts of succinic acid or anhydride monoesters or monoamides.

16. Composition according to claim 9, in which the polyolefin containing polar portions is an optionally modified polyisobutene containing succinic end groups.

17. Composition according to claim 9, in which the polyolefin containing polar portions is polyisobutene containing esterified succinic end groups.

18. Composition according to claim 9, comprising from 0.01% to 10% by weight of polyolefin(s) containing polar portion(s) relative to the total weight of the composition.

19. Composition according to claim 1, in which the emulsifying polymer comprises at least one amphiphilic polymer comprising at least one acrylamido-2-methylpropanesulfonic acid (AMPS) unit.

20. Composition according to claim 19, in which the emulsifying polymer is an amphiphilic polymer of at least one acrylamido-2-methylpropanesulfonic acid (AMPS) monomer and of at least one ethylenically unsaturated comonomer comprising at least one hydrophobic part containing from 7 to 30 carbon atoms.

21. Composition according to claim 20, in which the amphiphilic polymer comprises at least one ethylenically unsaturated hydrophilic comonomer chosen from acrylic acid, methacrylic acid or alkyl-substituted derivatives thereof or esters thereof obtained with monoalkylene or polyalkylene glycols, acrylamide, methacrylamide, vinylpyrrolidone, vinylformamide, maleic anhydride, itaconic acid or maleic acid.

22. Composition according to claim 20, in which the hydrophobic part is chosen from saturated or unsaturated linear alkyl radicals, branched alkyl radicals, and cyclic alkyl radicals.

23. Composition according to claim 21, in which the said ethylenically unsaturated hydrophobic comonomer is chosen from the acrylates or acrylamides of formula (1) below: 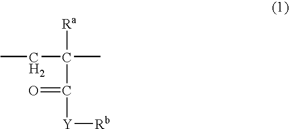
in which:
R^{a} denotes a hydrogen atom or a linear or branched C₁-C₆ alkyl radical;
Y denotes O or NH;
R^{b} denotes a hydrophobic radical comprising a fatty chain containing from 7 to 30 carbon atoms.

24. Composition according to claim 23, in which R^{b} is chosen from saturated or unsaturated linear C₇-C₂₂ alkyl radicals, branched alkyl radicals, or cyclic alkyl radicals, C₇-C₁₈ perfluoroalkyl radicals; the cholesteryl radical or a cholesterol ester, polycyclic aromatic groups.

25. Composition according to claim 18, in which the emulsifying polymer is chosen from crosslinked or non-crosslinked amphiphilic AMPS copolymers consisting of:
(a) 2-acrylamido-2-methylpropanesulfonic acid units of formula (2) below: 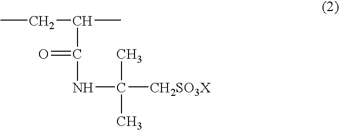
in which X is a proton, an alkali metal cation, an alkaline-earth metal cation or an ammonium ion; and
(b) units of formula (3) below: 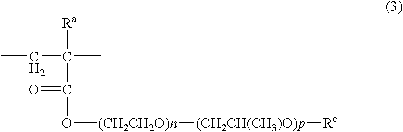
in which n and p, independently of one another, denote a number of moles and range from 0 to 30, with the proviso that n+p is less than or equal to 30, R^{a} denotes a hydrogen atom or a linear or branched C₁-C₆ alkyl radical, and R^{c} denotes a linear or branched alkyl from 7 to 22 carbon atoms.

26. Composition according claim 25, in which the emulsifying polymer is non-crosslinked, and is chosen from copolymers for which p=0, n=7 or 25, R^{a} denotes a methyl and R^{c} represents a mixture of C₁₂-C₁₄ or C₁₆-C₁₈ alkyl.

27. Composition according to claim 25, in which the emulsifying polymer is crosslinked, and is chosen from copolymers for which p=0, n=8 or 25, R^{a} denotes a methyl and R^{c} represents a mixture of C₁₆-C₁₈ alkyl.

28. Method for preventing and/or combating the signs of ageing of the skin comprising at least the step of applying to the skin a combination of at least one C-glycoside derivative and of at least one emulsifying polymer chosen from polyolefins containing polar portion(s) and amphiphilic polymers comprising at least one acrylamido-2-methylpropanesulfonic acid (AMPS) unit.

29. Method for making up and/or caring for keratin materials comprising at least the step of applying to the said keratin materials at least one coat of a composition as defined according to claim 1.
